(19) United States
(12) Patent Application Publication
(10) Pub. No.: US 2003/0119050 A1
(43) Pub. Date: Jun. 26, 2003

(54) Flow cytometer for analysis of general diagnostic factors in cells and body fluids

(76) Inventor: Shafrira Shai, Kiryat Haim (IL)

Correspondence Address: SOL SHEINBEIN, c/o ANTHONY CASTORINA, SUITE 207, 2001 JEFFERSON DAVIS HIGHWAY, ARLINGTON, VA, 22202, US

(21) Appl. No.: 10/354,203

(22) Filed: Jan. 30, 2003

Related U.S. Application Data
(62) Division of application No. 09/759,531, filed on Jan. 16, 2001.
Application No. 09/759,531 is a continuation of application No. 09/232,677, filed on Jan. 19, 1999, now abandoned.

(30) Foreign Application Priority Data
Mar. 30, 1998 (IL) 123891

(51) Int. Cl. C12Q 1/68; G01N 33/53; C12N 5/06; C12N 5/16

(52) U.S. Cl. 435/6; 435/345; 435/7.1

(57) ABSTRACT

The present invention relates to a system to analyse general diagnostic factors in cells and body fluids using a flow cytometer, and in particular to a system featuring a number of different fertility tests, in a simple, expedited format, in order to investigate factors affecting fertility, preferably in a semi or fully automated manner. Specifically, a preparative method has been developed to increase the success of in vitro fertilisation (I.V.F) and intrauterine insemination (I.U.I) in cases of immunoinfertility by removing sperm-bound antibodies from sperm cells. A special device has been designed to collect only motile sperm cells from semen samples. Thus, this invention provides improved methods for general diagnostic testing and infertility screening and enables gynecologists to obtain information from an infertile couple in a preliminary test, which until now has been time consuming and only possible to run in sophisticated laboratories.

### FIELD OF THE INVENTION

The present invention relates to analysis of general diagnostic factors in cells and body fluids using a flow cytometer, and in addition to a system featuring a number of different fertility tests, in a simple, expedited format, in order to investigate factors affecting fertility, preferably in a semi or fully automated manner. The same system can be used for other types of analysis, either in conjunction with fertility tests or as diagnosis of other conditions, such as for measurement of hormone levels in cells and body fluids. In particular, a preparative method has been developed to increase the success of in vitro fertilisation (I.V.F) and intrauterine insemination (I.U.I) in cases of immunoinfertility by removing sperm-bound antibodies from sperm cells. Also, a special device has been designed to collect only motile sperm cells from semen samples.

### BACKGROUND OF THE INVENTION

Approximately 10% of the adult population (ages 18-55) are infertile. Preliminary tests for the causes of infertility include checking the quality of the sperm sample from the male partner (volume, cell count, motility and morphology), and analysing the hormonal profile of the female partner. Other factors affecting fertility include infections of the genital tract such as Chlamydia trachomatis, and the presence of antisperm antibodies, bound to the sperm cells and in the neck of the cervix. The biological functionality of sperm may also determine fertility, in terms of the ability of the sperm to bind to components of the outer coat of the oocyte.

General Sperm Analysis

The analysis of sperm, including performing a sperm count, characterizing motility, viability, and sperm morphology, can provide useful information not only with respect to reproduction, but as an early warning monitor of exposure to dangerous agents into the body. Two parameters commonly used by urologists to measure fertility are sperm count and sperm motility. Sperm motility is defined as the fraction of sperm moving among all the sperm in a given specimen sample. The assessment of motile sperm fraction (total number of sperm cells of superior motility) can provide diagnostic information, which can direct the therapeutic approach. For the most part sperm motility and mean sperm velocity are simply estimated by visual examination of a drop of semen on a slide. The results of such visual examinations vary widely from one observer to another. Identification of various sperm precursor cells and somatic cells sometimes present in semen is also difficult. Furthermore linearity or velocity distribution functions cannot be estimated, purely on a visual examination.

Previous attempts have been made to automate these diagnostic tests. For example, U.S. Pat. No. 4,559,309 discloses a method by which RNA and DNA content/chromatin condensation as well as cell motility can all be determined using flow cytometry.

Another known method is based on the observation that a velocity-dependent frequency-modulated component is contained in the light scattered by the head section of spermia when the sperm sample is illuminated by the monochromatic light of a He-Ne laser. The velocity distribution of the spermia can be concluded by Fourier transformation of frequency spectrum of the Doppler signal. For example U.S. Pat. No. 4,880,732 discloses such a process.

In order to determine linearity or velocity distribution functions, a tedious method of multiple exposure time-lapse photography has been developed. This method requires the manual counting of the sperm tracks, followed by manual derivation of the distributions of linearity and velocity. In order to speed up this manual method, a computerised version has been developed, which allows for the calculation of the distribution functions, but only after the sperm tracks first have been manually outlined, by using an interactive indicating device such as a light pen. A further improved version employs a microscope attached to a computer, video recorder and other peripheral items. This improved version is designed to analyse a drop of semen in a special cell, called the Makler cell. The narrow spacing of the Makler cell, however, constricts the motion of the sperm tails. Therefore, a system employing the narrow Makler-type cell spacing adversely affects the very quantities that it is designed to measure. One version of this motility scanner is disclosed by Boisseau et al U.S. Pat. No. 4,896,966.

More recently, absorption spectrophotometry dye stained fluorometry, DNA determination and flow cytometry have been used to determine sperm count, while absorption spectrophotometry, time-lapse photography, cinematography and laser light-scattering have been used to determine sperm motility. U.S. Pat. No. 5,061,075 discloses measurement of the sperm count of a specimen of sperm by exciting the specimen with a beam of substantially monochromatic light, then measuring the intensity of the intrinsic native fluorescence emitted or the scattered light from the specimen and then determining the sperm count using the intensity measurements.

In recent decades the art has developed a very large number of protocols, test kits, and cartridges for conducting analyses on biological samples for various diagnostic and monitoring purposes. U.S. Pat. No. 5,427,946 discloses an analytical system which can analyse microvolumes of a sperm sample and produce analytical results rapidly. However, this device cannot be used to perform tests other than general sperm analysis.

Determination of Hormone Levels

(i) Non-fertility Hormones

Hormones can be divided into two main categories, water soluble hormones and lipid soluble hormones. Examples of water soluble hormones include insulin, growth hormone, TSH, FSH, LH and oxytocin. Lipid soluble hormones include cortisol, aldosterone, estrogen, progesterone, testosterone and thyroid hormone. Measurement of hormone levels in cells and body fluids (plasma, urine, saliva, seminal plasma) is a primary tool of the clinical endocrinologist.

The amount of hormones present in body fluid is usually measured with radio-immunoassays or ELISA assays. Immunometric assay kits for measurement of hormone levels are based on microtiter plates coated with a first antibody specific to the tested hormone. After reaction with the clinical samples, a second antibody specific to the hormone is added and the reaction is amplified by various systems (enzyme-substrate, biotein-avidin).

Although the measurement of hormone levels is a basic tool of routine clinical investigation, it has been methodologically complex. Firstly, the similar structure of hormones leads to significant problems with cross-reactivity. Secondly, most of the assays have been insufficiently sensitive. Thirdly, most commercial assays do not provide an adequate normative data base with which to compare patient samples (the normative data can vary with gender, age and developmental status).

(ii) Fertility Hormones

The female reproductive cycle is controlled by a number of different hormones, whose concentration alters throughout the monthly cycle. In order for pregnancy to be achieved and maintained these hormones must remain in balance. One example of such a hormone is luteinising hormone (LH). One of the objectives of measuring the luteinising hormone is to determine the ovulation time point in the case of an induction of pregnancy. For the determination of LH, there are especially suitable immunological test processes, in which the hormone is determined as antigen with one or more antibodies directed against it. The preparation of antibodies with these polypeptide hormones involves difficulties since all polypeptide hormones are poorly immunogenic. An antibody directed against one of the glycoprotein hormones, e.g., follicle-stimulating hormone (FSH), thyreotropin-stimulating-hormone (TSH) and human chorionic gonadotropin (hCG) usually displays more or less cross-reactivity with the other glycoprotein hormones. A monoclonal antibody which is specifically directed against LH and displays no cross-reactivity is not yet known. U.S. Pat. No. 5,2248,593 discloses an immunological process and reagent to specifically determine LH levels even in the presence of other glycoprotein hormones. U.S. Pat. No. 4,762,783 also discloses an immunological process for the determination of the follicle-stimulating-hormone (FSH). However, these tests have the drawback of requiring substantial manual intervention.

Prediction of the Success of In Vitro Fertilisation

The technique of IVF has been used in human patients with infertility problems successfully since 1978. Despite extensive research it is still a difficult procedure and even in the best IVF clinics a success rate of only 30% is generally achieved. Surgical procedures are required to collect eggs for IVF and further surgery is required to implant fertilized eggs in the womb. The recipient must then wait for a period of time before it can be determined whether or not pregnancy has been established. In some cases, pregnancy may never be established despite numerous attempts representing a considerable expense to society. Additional problems include the occurrence of multiple pregnancies, the increase of perinatal mortality and the late consequences of low birth weight.

When several ova are removed from the ovaries of a woman, visual examination is not sufficient to determine if a particular ovum was taken from a healthy follicle and is likely to undergo fertilisation, or if it is from an atretic follicle. In consequence, when in vitro fertilisation is being utilised usually several ova are removed from the follicles of the woman and fertilised.

The chances of a successful pregnancy would be increased by finding those ova having a high probability of potential fertilisation, to fertilise only these ova, and to implant only them. The conventional method to predict the success of fertilising an ovum taken from a human follicle involves an analysis of the follicular fluid in which the ovum has been bathed. The concentration of steroids in the follicular fluid are very low, making analysis of them very difficult. This method has therefore generally been limited to experimental situations. U.S. Pat. No. 4,772,554 discloses a method for assaying the fertilisation potential of a mammalian ovum that has been removed form an ovarian follicle, together with a portion of accompanying follicular fluid.

Identification of recipients for whom IVF is unlikely to be successful prior to treatment is desirable. U.S. Pat. No. 5,635,366 discloses that once fertilization has been achieved and the second part of the IVF procedure is performed, namely implantation, there is a strong inverse correlation between levels of 11β-HSD in the environment of the oocyte at the time of collection and the subsequent establishment of pregnancy. This correlation exists regardless of the maturity of the oocyte or other factors which may affect fertilization.

Reliable prognostic assays are needed to determine which infertile men are likely to achieve fertilisation in vivo or impregnate their female partners when assisted by artificial insemination. One example of such an assay for tight sperm binding to the mammalian hemizona pellucida is disclosed in U.S. Pat. No. 5,219,729.

Human spermatozoa binding to the human zona pellucida represents the first critical event in gamete interaction leading to fertilization and activation of development. This binding step may provide unique information predictive of ultimate sperm fertilising potential. Due to species specificity, human spermatozoa will bind firmly to only human zona pellucida.

Identification of Infections

Chlamydia Trachomatis

Chlamydia trachomatic 1 is one of two microbial species of the genus Chlamydiaceae, order Chlamydiales. There are fifteen or more serotypes of this species which are the causes of a number of human ocular and genital diseases. The majority of cervical infections are asymptomatic and, if untreated, may progress to pelvic inflammatory disease, which can result in infertility. Gonorrhea is a disease usually transmitted by sexual contact caused by a bacterium of the Neisseria genus. The importance of detection and treatment of this organism is well recognised. Antibiotics have helped control its spread, although it still persists in epidemic proportions in some parts of the world.

Currently accepted procedures for the detection of Chlamydial infection rely upon culture techniques. These techniques are time-consuming, expensive and subject to technician error. In addition to culture procedures, various immunoassay techniques for the detection of Chlamydial infection have been described. In order to accurately diagnose the presence of Chlamydial infection, it is preferred to assay for antigens rather than antibodies.

U.S. Pat. No. 4,497,899 discloses a solid phase immunoassay procedure for the detection of Chlamydia trachomatis antigens in a clinical specimen. The Chlamydia trachomatis antigens to be determined are coated or adsorbed on the solid phase. The coated antigen is then detected with either one or two antibodies, one of which is suitably labeled. This assay takes at least three hours to perform. A more rapid and reliable test describes the use of an ionically charged support that attracts Chlamydial or gonococcal antigens enabling their quick and sensitive detection. A further improvement is the use of a surfactant-coated uncharged membrane in Chlamydial assays. This allows detection of the antigen in biological specimens that contain copious amounts of whole blood, mucous or components thereof.

U.S. Pat. No. 4.916,057 discloses an immunoassay procedure for the detection of Chlamydia trachomatis antigen in a urogenital clinical specimen including a method for substantially eliminating the occurrence of false negative and false positive results of the immunoassay procedure.

U.S. Pat. Nos. 5,085,986 and 5,032,504 disclose a diagnostic test kit and method for determination of Chlamydial or gonococcal antigens.

U.S. Pat. No. 5,030,561 discloses a method for assaying of Chlamydia, which includes adhering Chlamydia antigen to amidine modified latex particles, binding of adhered antigen to an anti-Chlamydia antibody conjugated to an enzyme, separating the particles from the liquid phase of the assay and detecting bound enzyme by colour development when the separated particles are contacted with a substrate for the enzyme.

U.S. Pat. No. 5,188,937 discloses an assay for Chlamydia which includes contacting Chlamydia organisms in a liquid with a solid support having an antispecies Fc antibody immobilised thereon and an anti-Chlamydia capture antibody. After binding of Chlamydia antigen to the capture antibody and binding of the capture antibody to the antispecies antibody on the support, a tracer including a label conjugated to a signal antibody is added. After binding of the signal antibody to the antigen, the presence of Chlamydia organisms in the liquid is detected by a signal associated with the label thereby bound to the support.

Identification of Sperm Antibodies

Autoantigens are tissue components of an organism to which that organism directs an immune response. The condition which results from such a self-directed immune response is known as autoimmunity. Proteins on sperm are known to be potent autoantigens and autoimmunity to such proteins is believed a significant cause of infertility. One such protein, mammalian split protein, is disclosed in U.S. Pat. No. 5,616,322.

Sp-10 is a sperm-specific antigen identified as an acrosomal constituent present through spermiogenesis. A monoclonal antibody specific for this tissue-specific antigen has been previously developed, identified as MHS-10. U.S. Pat. No. 5,605,803 discloses a kit and method for detecting sperm production in a human male individual which includes this antibody.

Capacitation of Sperm

The medical community is often concerned with human fertility, but has few reliable methods for evaluating the fertility of male patients. For example, there is a lack of effective methods for detecting lack of capaciatation in the sperm of a patient. Mammalian spermatozoa in semen cannot fertilize eggs but must undergo alterations in the plasma membrane in order to acquire fertilizing capability. The process during which the spermatozoa undergo these alterations in the membrane is termed capacitation and occurs naturally in the female reproductive tract once the sperm has been deposited. Capacitation refers to the ability of sperm to adhere to, penetrate and fertilize susceptible ova. Successful capacitation of the sperm is widely considered to be one of the factors for determining the fertilizing capacity of the sperm of a test subject. U.S. Pat. No. 5,256,539 discloses diagnostic assays using antibodies to fibronectin to detect a lack of capacitation in a sample of human spermatozoa due to disorders related to fibronectin expression on the sperm surface.

U.S. Pat. No. 5,389,519 discloses a method for detecting infertility in mammalian male subjects, by measuring capacitation in a sample of sperm with one or more monoclonal or polyclonal antibodies directed against a specific polypeptide.

There is thus a widely recognized need for, and it would be highly advantageous to have kits for automatically performing analysis of general diagnostic factors and fertility factors in cells and body fluids without the need of highly sophisticated and expensive clinical laboratory equipment as is described in the present invention.

### SUMMARY OF THE INVENTION

The present invention provides a system to analyse general diagnostic factors in cells and body fluids using a flow cytometer, and in particular to a system featuring a number of different fertility tests, in a simple, expedited format, in order to investigate factors affecting fertility, preferably in a semi or fully automated manner. Additionally, the same system can be used for more general analysis, such as for measurement of hormone levels and concentration of autoantibodies and infectious agents in cells and body fluids.

A fertility kit determines at least one fertility affecting factor and is used to perform a fertility test. One cervical smear, one semen sample and one serum sample from each member of the couple are preferably sufficient for substantially all tests. A cervical smear is defined as a sample taken from the cervix of the female partner. A plurality of tests can be performed on a single sample. Each test includes at least one reagent. The reagent is able to react with the sample to form a reaction product and a flow cytometer is able to analyse the reaction product to determine the fertility factor.

Alternatively, a kit can determine a diagnostic factor from a sample of cells and body fluids, such as a non-fertility hormone level. A plurality of tests can be performed on a single sample. Each test includes at least one reagent. The reagent is able to react with the sample to form a reaction product and a flow cytometer is able to analyse the reaction product to determine the diagnostic factor.

The term ‘general diagnostic factors’ as used herein refers to hormone levels and antigens to any component of an infectious agent.

Specifically these tests include the assessment of the sperm sample (sperm count, motility, morphology, viability, white blood cells and sperm-bound antibodies), the identification of sperm antibodies on the sperm cells and in the neck of the cervix of the female, the identification of infectious agents including infectious agents known to affect fertility, such as Chlamydia in both sperm and cervical samples, the determination of hormone levels, including Luteinizing Hormone (LH), Follicle Stimulating Hormone (FSH) or Testosterone levels in the serum sample of each member of the couple, and the assessment of the ability of sperm to attach to peptides taken from the outer coat of the oocyte and the ability of sperm cells to undergo acrosome reaction and DNA stability. The results of these tests may be used for predicting success of I.U.I and IVF treatment and subsequently determine approval or disapproval of I.V.F and I.U.I treatment. In addition, a preparative method has been developed to increase the success of I.V.F and I.U.I, in case of antisperm antibodies where sperm bound antibodies and white blood cells are removed from semen. A novel device has been designed to collect only motile sperm cells from the semen sample.

The assessment of sperm quality includes tests to determine sperm motility, viability and morphology with fluorescent dyes. Sperm count is calculated using a flow cytometer.

The detection of infectious agents utilises tests for the presence of chlamydial, gonoccal organisms and mycoplasma. Levels of various reproductive components in samples taken from sera of the couple are determined. This includes tests for the presence of LH, FSH and testosterone in serum samples. These tests are based on the binding of specific monoclonal antibodies to infectious agents or hormones to cells and body fluid beads and reacting them with the test sample. A second monoclonal antibody, specific for the infectious agent or hormone and biotin labeled, is applied to direct the binding of fluorescent streptavadin to the beads. The same method can be used for determining other hormone levels.

Testing of sperm auto-antibodies is considered to be an integral part of the initial semen evaluation. A novel solution to remove antisperm antibodies from sperm cells without interfering with cell function has been developed and can be applied to increase success rate of I.V.F and I.U.I in relevant cases. In vitro bioassay of spermatozoa to determine the ability of sperm to bind to the zp-3 (zona pellucida 3 antigen) of the oocyte together with the ability of sperm cells to undergo acrosome reaction will help to direct those cases without evidence of sperm zp binding, straight to intracytoplasmic sperm injection (ICSI) treatment, where the binding of spermatozoa to zp is not necessary. The test is based on the binding of sperm cells to fluorescent micro sphere beads such as latex beads coated with peptides of zp-3.

According to the teachings of the present invention there is provided in a first embodiment a semi-automated fertility system for assessing the fertility of a couple, the couple consisting of a male partner and a female partner, comprising

(a) a cervical smear including cervical mucus and at least one serum sample from the female partner;

(b) at least one semen sample and at least one serum sample from the male partner;

(c) a fertility kit for determining at least one fertility affecting factor, the fertility kit being used to perform a fertility test, the fertility kit including at least one reagent, such that the reagent is able to react with a sample selected from the group consisting of a cervical smear and serum sample from the female partner and a semen sample and a serum sample from the male partner, to form a reaction product and

(d) a flow cytometer, such that the flow cytometer is able to analyse the reaction product to determine the fertility factor.

In a preferred embodiment a plurality of tests can be performed on a single sample of the group consisting of, at least one female cervical smear, female cervical mucus, at least one female serum sample, at least one male semen sample and at least one male serum sample.

In a preferred embodiment the sample from the male partner is the semen sample, and the reagent is a viscous solution, such that motility of sperm in the sample is determined according to movement of the sperm through the viscous solution.

In a preferred embodiment the viscous solution includes a dye.

In a preferred embodiment the system further comprises a device for measuring sperm motility in a sample of sperm, the device comprising, a sample compartment, at least one channel and a barrier separating the sample compartment from the at least one channel, such that the sperm must cross over the barrier from the sample compartment to reach the at least one channel.

In a preferred embodiment the sample from the male partner is the semen sample, and the reagent is a dye to identify live cells, such that the fertility test determines a number of live cells.

In a preferred embodiment the dye includes dichlorfluorescein.

In a preferred embodiment the sample from the male partner is the semen sample, and the reagent is a morphology gate system comprising at least one gate such that the fertility test determines sperm cell morphology according to an ability of the sperm cells to enter through the at least one gate.

In a preferred embodiment the access is determined by geometry of the gate.

In a preferred embodiment the system to determine cell morphology further comprises a dye.

In a preferred embodiment the dye is acridine orange.

In a preferred embodiment the sample from the male partner is the semen sample, and the reagent comprises:

(a) a solution including anti human antibodies conjugated with fluorescent dye, the anti human antibodies binding to an antibody present in cells of the semen sample; and

(b) a second solution including a dyed label, the dyed label binding to the anti human antibodies, such that antibodies bound to sperm are detected and such that the fertility test is detection of sperm-bound antibodies.

In a preferred embodiment the reagent comprises a solution to remove non-specific antibodies and a second solution to block non-specific antibody binding sites on the sperm surface.

In a preferred embodiment the sample from the male partner is the semen sample, and the reagent is fluorescent micro sphere beads coated with zp-3 peptides and the fertility test is ability of the sperm to bind to the beads.

In a preferred embodiment the sample comprises the cervical smear of the female partner and the semen sample of the male partner, and the reagent comprises at least one antibody specific to at least one infectious agent of the genitalia, such that the fertility test is detection of the infectious agent in the cervical smear and semen sample.

In a preferred embodiment the system further comprises, polystyrene micro sphere beads coated with an antibody specific to an infectious agent, at least one biotin labeled antibody specific to the infectious agent, the biotin conjugate binding to the beads, a streptavidin protein, the protein binding to biotin and a fluorescent labeled dye, binding to the antibody.

In a preferred embodiment the sample comprises the cervical smear of the female partner and the semen sample of the male partner, and the reagent comprises at least one antibody specific to Clamydia trachomatis, such that the fertility test is detection of Chlamydia trachomatis in cervical smear and semen sample.

In a preferred embodiment the system further comprises, polystyrene micro sphere beads coated with an antibody specific to Clamydia trachomatis, at least one biotin labeled antibody specific to Chlamydia trachomatis, the biotin labeled antibody binding to the beads, a streptavidin protein binding to biotin and a fluorescent labeled dye binding to the antibody.

In a preferred embodiment the sample comprises the serum sample of the female partner and the serum sample of the male partner such that the fertility test is detection of hormone levels in serum sample.

In a preferred embodiment the reagent further comprises at least one polystyrene micro sphere bead coated with antibodies specific for the hormone to be tested, at least one biotin labeled antibody binding to the hormone, a streptavidin protein binding to biotin and a fluorescent labeled dye binding to the antibody.

In a preferred embodiment the fertility test is the ability of sperm cells to undergo acrosome reaction.

In a preferred embodiment the fertility test is sperm cell count and white blood cell count.

In a second embodiment the invention provides a semi-automated system for assessing diagnostic factors, comprising;

(a) at least one cell and body fluid sample;

(b) a kit for determining at least one diagnostic factor, the kit being used to perform a diagnostic test, the kit including at least one reagent, such that the reagent is able to react with at least one cell and body fluid sample to form a reaction product and

(c) a flow cytometer, such that the flow cytometer is able to analyse the reaction product to determine the diagnostic factor.

In a preferred embodiment the diagnostic factor is hormone level.

In a preferred embodiment the diagnostic factor is the identification of antigens of any component of an infectious agent.

In a preferred embodiment the diagnostic factor is a fertility factor.

In a third embodiment the present invention provides a method for detecting sperm-binding antibodies in cervical mucus of the female partner comprising the steps of:

(a) washing semen sample of the male partner in a solution of low pH to remove specific and non specific antibodies;

(b) incubating the semen sample of the male partner in a solution to block non specific binding sites in the serum sample;

(c) incubating treated semen sample of the male partner with cervical mucus of the female partner;

(d) incubating mixture of the treated semen sample of the male partner and cervical mucus of the female partner with anti human antibodies bound to fluorescent dye, and

(e) detecting results in flow cytometer.

In a fourth embodiment, the present invention provides a method for predicting success of IVF and IUI treatment, comprising the steps of:

(a) washing and capacitation of sperm sample,

(b) incubating the sperm sample with fluorescently labeled beads coated with peptides of the oocyte- membrane,

(c) washing the sperm cells and

(d) detecting sperm cells bound to the oocyte membrane peptide to predict success of IVF and IUI treatment.

In a preferred embodiment the prediction of success of IVF and IUI treatment is determined by visual observation of a dye.

In a fifth embodiment, the present invention provides a method of collecting motile sperm cells from a sample of sperm, comprising the steps of:

(a) providing a device for measuring sperm motility in a sample of sperm, the device including;

(i) a sample compartment,

(ii) at least one channel and

(iii) a barrier separating the sample compartment from the at least one channel, such that the sperm must cross over the barrier from the sample compartment to reach the channel,

(b) filling the channels of the device with a viscous solution,

(c) putting the sample in the sample compartment of the device and

(d) collecting motile sperm cells from the channels of the device.

In a preferred embodiment the method of collecting motile sperm cells from a sample of sperm further comprises separating white blood cells by magnetic separation with magnetic beads coated with anti CD-45 antibodies.

In a sixth embodiment, the present invention provides a method of removal of sperm bound antibodies from semen comprising the steps of:

(a) forming a cell pellet by centrifugation of the semen,

(b) adding an acidic solution to the cell pellet to remove antisperm antibodies and

(c) resuspending cell pellet in a mixture of washing solution, reagent to increase cell motility and a reagent to prevent free radical production to obtain semen without sperm bound antibodies.

In a preferred embodiment the reagent to increase cell motility includes hyaluronic acid.

In a preferred embodiment the reagent to prevent free radical production includes ferulic acid.

In a seventh embodiment, the present invention provides a method for increasing success of IVF treatment and IUI treatment, comprising the steps of:

(a) removing white blood cells and separating motile sperm cells from semen by:

(i) providing a device, for separation of motile sperm cells from non-motile material, the non-motile material including white blood cells, in a sample of sperm, the device comprising;

(I) a sample compartment,

(II) at least one channel and

(III) a barrier separating the sample compartment from the at least one channel, such that the sperm must cross over the barrier from the sample compartment to reach the channel;

(ii) filling the channels of the device with a viscous solution;

(iii) mixing semen with magnetic beads coupled with anti CD45;

(iv) putting the sample in the sample compartment and incubating and

(v) collecting motile sperm cells from the channels;

(b) removing sperm bound antibodies by:

(i) forming a cell pellet by centrifugation;

(ii) adding an acidic solution to remove antisperm antibodies and

(iii) resuspending cell pellet in a mixture of washing solution, reagent to increase cell motility and a reagent to free radical production.

In an eighth embodiment, the present invention provides a device for measuring sperm motility in a sample of sperm, comprising;

(a) a sample compartment;

(b) at least one channel and

(c) a barrier separating the sample compartment from the at least one channel, such that the sperm must cross over the barrier from the sample compartment to reach the at least one channel.

In a preferred embodiment the at least one channel contains a viscous fluid.

In a preferred embodiment the viscous fluid contains at least one dye, such that the sperm are able to contact the dye upon reaching the at least one channel.

### BRIEF DESCRIPTION OF THE DRAWING

FIG. 1A shows a flow chart of the in parallel analysis of several fertility factors using a flow cytometer like instrument according to the present invention.

FIG. 1B shows in parallel analysis of general diagnostic factors in cells and body fluids.

FIG. 2 shows an exemplary device for determining sperm motility according to the present invention.

FIG. 3 shows analysis of the motility of three serum samples.

FIG. 4 shows analysis of the morphology of two sperm cell samples.

FIG. 5 shows analysis of the percentage of tested sperm cells bound with ZP-3.

FIG. 6 shows analysis of the percentage of tested sperm cells that underwent acrosome reaction.

FIG. 7 shows identification of ZP-3 autoantibodies in tested female sera.

FIG. 8 shows a graph depicting levels of sperm-bound antibody IgG, from sperm cells in five patients before and after treatment to remove sperm-bound antibodies.

FIG. 9 shows a graph depicting levels of sperm-bound antibody IgA, from sperm cells in five patients before and after treatment to remove sperm-bound antibodies.

### DETAILED DESCRIPTION OF THE PREFERRED EMBODIMENTS

The present invention provides a system to analyse general diagnostic factors in cells and body fluids using a flow cytometer, and in particular to a system featuring a number of different fertility tests, in a simple, expedited format, in order to investigate factors affecting fertility, preferably in a semi or fully automated manner. Additionally, the same system can be used for more general analysis, such as for measurement of hormone levels, concentration of autoantibodies and infectious agents in cells and body fluids.

A fertility kit determines at least one fertility affecting factor and is used to perform a fertility test. One cervical smear, one semen sample and one serum sample from each member of the couple are preferably sufficient for substantially all tests. A cervical smear is defined as a sample taken from the cervix of the female partner. A plurality of tests can be performed on a single sample. Each test includes at least one reagent. The reagent is able to react with the sample to form a reaction product and a flow cytometer is able to analyse the reaction product to determine the fertility factor.

Alternatively, a kit can determine a diagnostic factor from a sample of cells and body fluids, such as a non-fertility hormone level. A plurality of tests can be performed on a single sample. Each test includes at least one reagent. The reagent is able to react with the sample to form a reaction product and a flow cytometer is able to analyse the reaction product to determine the diagnostic factor.

A description of each kit is given under the individual headings below. Each kit will contain the relevant protocol, solutions, reagents and controls. The kits include:

1. General Analysis of semen (sperm count, sperm motility, sperm morphology, viability, white blood cells, immature sperm cells and sperm-bound antibodies in sperm sample and in neck of the cervix of the female).

2. Prediction of in-vitro fertilization success-acrosome reaction, binding capabilities to ovum and DNA stability.

3. Identification of infection such as genital infection by Chlamydia Trachomatis in the semen and in the neck of the cervix.

4. Evaluation of hormone levels, such as LH, FSH, testosterone, progesterone, beta-estradiol and prolactin.

5. IVF-sperm pretreatment—this kit removes sperm bound antibodies and white blood cells from semen before IVF, IUI and cryopreservation of spermatozoa.

6. Immunofertility—antibodies to: ovarian, zona pellucida, sperm, LH, FSH, phospholipids and inhibin.

Furthermore, a novel device to enable easy separation of motile sperm cells from the sample has also been designed.

An instrument similar to a flow cytometer enables automation of these tests. Semi-automation includes full automation, whereby the entire reading or substantially the whole method is conducted by machine, as well as semi-automation, which can include reading both manually and by machine or preferably at least part of the method of the test being conducted by machine or manually.

The present invention overcomes the shortcomings of the background art by providing simple automation of these diagnostic and fertility tests in an instrument similar to a flow cytometer. Additionally, a novel device enables easy separation of motile sperm cells from the sample. Furthermore, the present invention identifies antisperm antibodies in cervical mucus using the male partner's sperm. The present invention tests the biological function of sperm cells to bind to the oocyte, without using actual, whole oocytes and in-vitro tests the ability of sperm cells to undergo acrosome reaction. The invention provides a novel method for a fertility aid to remove sperm bound antibodies and white blood cells from semen. The described method is easier to perform and requires less washing steps, subsequently keeping the sperm cells in better condition than existing known methods. A novel application of the flow cytometer is precise measurement of hormone levels as is described in the present invention, which was neither taught nor suggested by the prior art. The present invention tests all the parameters that are recommended by the WHO for general analysis of semen using a flow cytometer, which gives quantitative results and does not rely on observations by eye. There is also provided a method of identifying infectious agents in cells and body fluids using a flow cytometer and the like. This method is another novel application of the flow cytometer. An additional novel application of the flow cytometer is its use in determining the existence of autoantibodies in body fluids.

The main users of these kits will be hospital research laboratories and fertility clinics. Gynecologists will be able to obtain information from an infertile couple in a preliminary test, which until now has been time consuming.

The present invention provides a system to analyse general diagnostic factors in cells and body fluids using a flow cytometer, and in particular to a system featuring a number of different fertility tests, in a simple, expedited format, in order to investigate factors affecting fertility, preferably in a semi or fully automated manner. The present invention may be better understood with reference to the figures. The figures show one embodiment of the present invention and are not limiting.

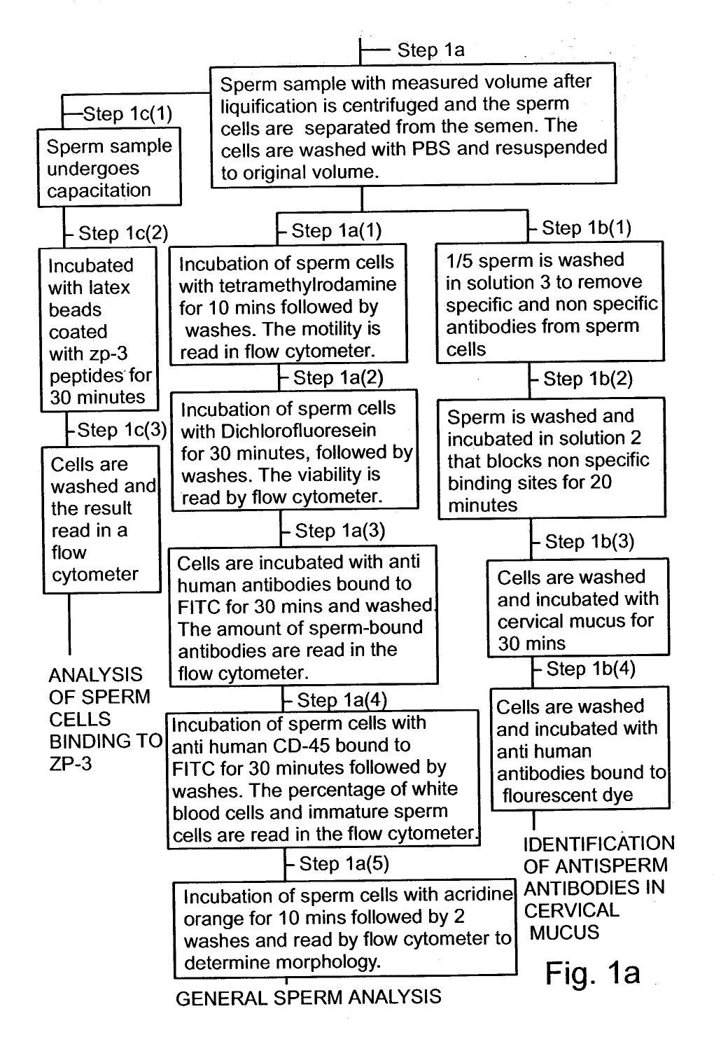

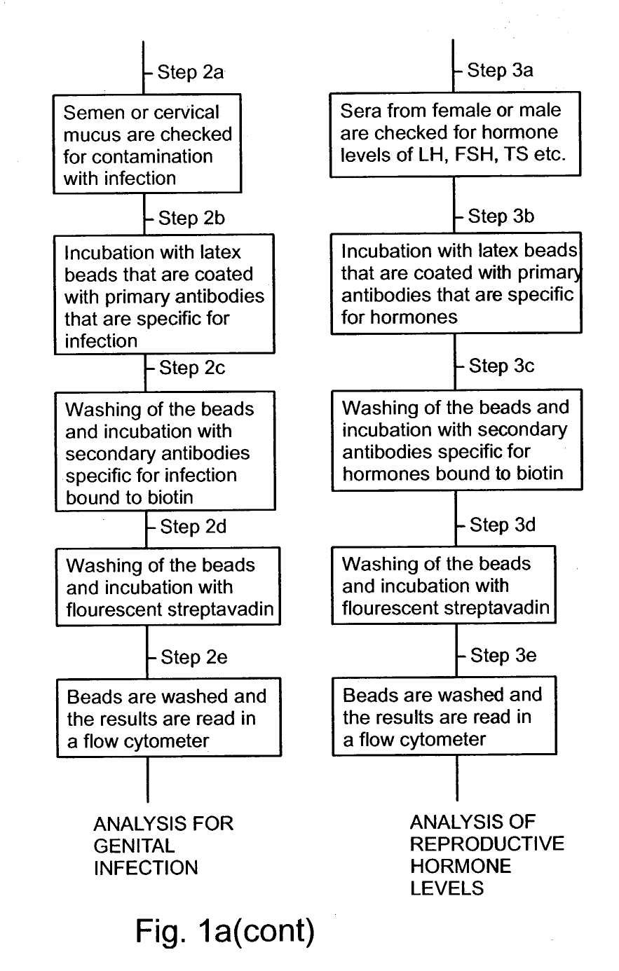

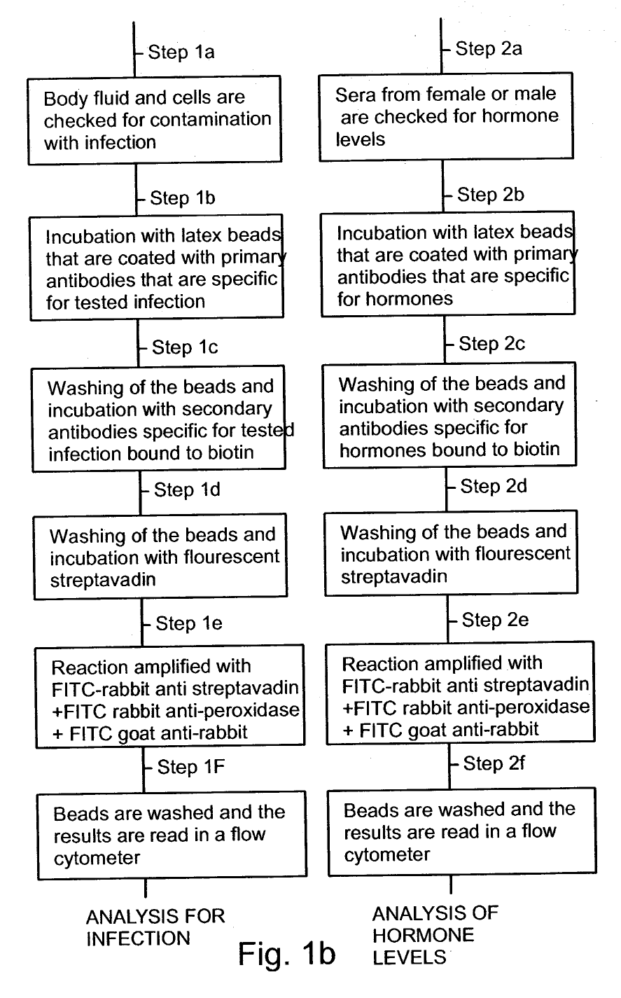

The following steps as shown in FIG. 1A describe the simultaneous analyses of the sperm sample and are as follows.

General Sperm Analysis

In step 1a part of the sample is taken for general analysis. Sperm sample with measured volume after liquification is centrifuged and the sperm cells are separated from semen. The cells are washed with PBS and resuspended to original volume. In step 1a(1)the sperm cells are incubated with tetramethylrodamine for about 10 minutes, followed by washes. The motility is read in a flow cytometer. In step 1a(2) the sperm cells are incubated with dichlorofluoresein for about 30 minutes, followed by washes. The viability can then be read by flow cytometer. In step 1a(3) cells are incubated with anti human antibodies bound to FITC forabout 30 minutes and washed. The amount of sperm-bound antibodies are then read in the flow cytometer. Subsequently, in step 1a(4) sperm cells are incubated with anti human CD-45 bound to FITC for about 30 minutes, followed by washes. The percentage of white blood cells and immature cells are then read in the flow cytometer. In step 1a(5) sperm cells are incubated with acridine orange for 10 minutes, followed by washes. Morphology is then determined by flow cytometer.

Device for the removal of white blood cells from semen and determining sperm motility

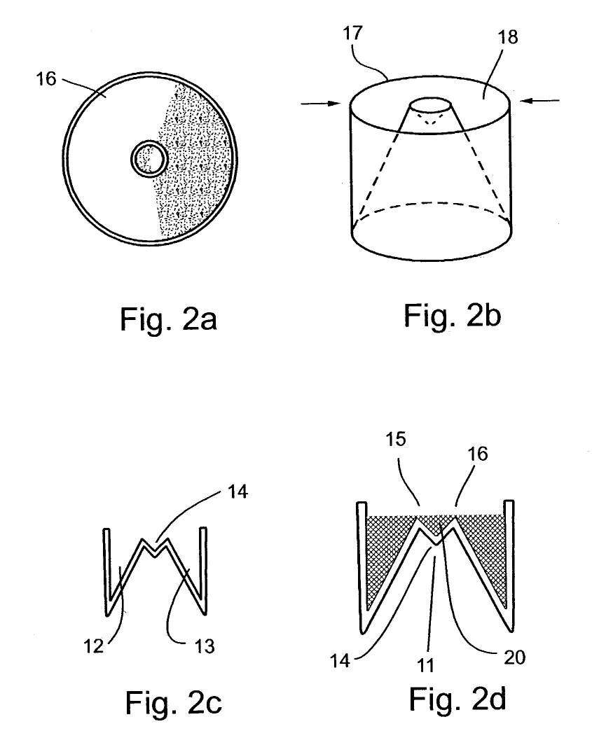

FIGS. 2A-D show a novel device 10 for determining sperm motility, incorporating a specially designed assay for determining sperm motility. FIG. 2A shows the device from a top view, FIG. 2B shows a partial cutaway view from the side, FIG. 2C depicts a full cutaway view from the side with the device empty and, FIG. 2D depicts a full cutaway view from the side of the device containing fluid. Device 10 has a lip 17 around a central chamber 18, two side channels 12 and 13 and a central sample compartment 14. Fluid containing viscous medium of a suitable volume such as 1-2 ml of Ficoll and a dye is poured into the two side channels 12 and 13 of the device. In the central sample compartment 14, a glass wool filter 11 is placed, in order to absorb dead cells and white blood cells. After seminal liquidation, seminal plasma is separated from cells by centrifuging and resuspension of the cell pellet, with a suitable volume which for a typical sample size is 0.5 ml of a neutral solution such as 0.15M Hepes at pH 7.2. The sperm sample is washed and reduced to a suitable volume for example 0.5 ml and placed in the central sample compartment 14 of the device. The fluid touches the sperm sample at points 15 and 16. The motile sperm will move into the viscous fluid, whereas the immobile sperm cannot cross over the barriers 19 and 20 separating the sample compartment 14 and side channels 12 and 13. Subsequently the motile sperm can be separated from the sperm sample. The sample is incubated for a suitable time under suitable conditions such as one hour at 37° C. Solution is then collected from both side channels 12 and 13 and this is the motile fraction.

Identification of antisperm antibodies in cervical mucus:

A sperm sample of measured volume, approximately ⅕ part of the sample, undergoes a set of tests. The set tests the presence of antisperm antibodies in cervical mucus. The sample is liquified and centrifuged and the sperm cells are separated from the semen. The cells are washed with PBS and resuspended in PBS to the original volume. The sperm solution is passed through glass wool to remove white blood cells and dead cells. This sample undergoes the following pathway (FIG. 1A).

Identification of antisperm antibodies in cervical mucus

In the pathway (FIG. 1A) to test the presence of antisperm antibodies in cervical mucus, the sperm is washed in a solution to remove specific and non specific antibodies (step 1b(1)). In step 1b(2) the sperm is washed and incubated in a solution that blocks non specific binding sites. Cells are then washed and incubated with liquified cervical mucus from the neck of the cervix of the female (step 1b(3)). In step 1b(4) cells are washed and incubated with anti-human antibodies bound to fluorescent dye and after 30 minutes incubation the cells are washed and the results read in a flow cytometer. This method has the advantage of using the male partner's sperm, unlike currently available background art methods, which rely upon sperm taken from donors.

Analysis of sperm cells ability to bind to zp-3

The ability of sperm cells to bind to zp is tested. Sperm cells undergo capacitation (FIG. 1A) in step 1c(1) and are incubated with beads such as fluorescent microsphere beads coated with zp-3 peptides for about 30 minutes in step 1c(2). In step 1c(3) cells are washed and the results read in a flow cytometer. Capacitation in the biological sense is a physiological process, whereby the spermatozoa undergo changes to acquire fertilising capability once the sperm has been deposited in the female reproductive tract.

Analysis for Chlamydia Trachomatis infection

Seminal plasma or cervical mucus are checked (FIG. 1A) for contamination with Chlamydia Trachomatis (step 2a). This sample is then incubated with micro sphere beads such as latex beads that are coated with primary antibodies that are specific for Chlamydia Trachomatis (step 2b). In step 2c the beads are washed and incubated with secondary antibodies specific for Chlamydia Trachomatis bound to biotin. The beads are then washed and incubated with fluorescent streptavadin (step 2d) and in step 2e the beads are washed and the results are read in a flow cytometer.

Analysis of fertility hormone levels

Serum is checked (FIG. 1A) for hormone levels of LH, FSH and TH (step 3a). This sample is then incubated with micro sphere beads such as latex beads that are coated with primary antibodies that are specific for hormones (step 3b). In step 3c the beads are washed and incubated with secondary antibodies specific for hormones bound to biotin. The beads are then washed and incubated with fluorescent streptavadin (step 3d) and in step 3e the beads are washed and the results are read in a flow cytometer.

Analysis of the concentration of one or more hormones as the diagnostic factor

Sera from female or male are checked for hormone levels (FIG. 1B, step 2a). Beads coated with primary antibodies to the tested hormone are incubated with cells and body fluids (step 2b) for 1 hour, the beads are then washed and incubated with biotin labeled monoclonal antibodies highly specific for the tested hormone (step 2c). After washing, fluorescent streptavidin that has high affinity to biotin is added (step 2d). The reaction is then amplified with FITC-rabbit anti streptavidin and FITC-rabbit anti peroxidase and FITC-goat anti rabbit. (step 2e). The results are then read by flow cytometer and then analysed by special software (step 2f).

Identification of infection by analysis of one or more antigens to any component of an infectious agent as the diagnostic factor

Body fluid and cells are checked for contamination with infection (FIG. 1B, step 1a). Beads coated with primary antibodies to the tested infection are incubated with cells or body fluids (step 1b) for 1 hour, the beads are then washed and incubated with biotin labeled monoclonal antibodies highly specific for the tested infection (step 1c). After washing, fluorescent streptavidin that has high affinity to biotin is added (step 1d). The reaction is then amplified with FITC-rabbit anti streptavidin and FITC-rabbit anti peroxidase and FITC-goat anti rabbit (step 1e). The results are then read by flow cytometer and then analysed by special software (step 1f).

Analysis of sperm sample

Cell count of sperm sample is done by preparation of three control standards of micro sphere beads such as latex beads, in which each standard has a known number of beads and their reading can be compared to the cell count. The cell count is done automatically by a flow cytometer and dead cells and non semen material are separated by the machine and are not analysed. This is done by the size of the cell or presence of a dye that is absorbed by the dead cells. The cell count is an average of three readings.

Cell motility is checked by placing a drop of the test sample in a novel device for determining sperm motility surrounded by a viscous solution (e.g. Ficoll), containing fluorescent dye that passively crosses the cell membrane and stays inside the cell by interaction with cell enzymes. Only mobile cells will penetrate into the viscous solution, and the greater the content of dye that is absorbed, the faster the cell. After an interval of time, the cells are collected in a tube and washed. The percentage of dyed cells that are counted by the flow cytometer is the percentage of cell motility. A test of normal cell morphology is conducted using a morphology gate system, with specific criteria that will define a normal cell (mainly parameters of size and shape), whereby access of the cell through the gate is determined by geometry (size and shape) of the gate. Cells which are non standard will be read as abnormal.

Removal of sperm bound antibodies and white blood cells from semen

Sperm bound antibodies and white blood cells from semen need to be removed before In Vitro Fertilisation (IVF), intrauterine insemination (IUI), and cryopreservation of spermatozoa. The white blood cells are removed by magnetic separation after incubation of semen with magnetic beads coated with antibodies to white blood cells. The sperm cells are washed to remove antibodies from sperm cells, followed by further washing of the sperm cells after treatment to keep the cells.

The examples and descriptions are intended only to serve as examples, and many other embodiments are possible within the spirit and the scope of the present invention.

### EXAMPLE 1

Specific example of general analysis of sperm sample

General analysis of the sperm sample specifically measures cell count, percentage and number of motile cells, normal morphology, number and percentage of white blood cells and number and percentage of dead cells. A number of tubes are used in the analysis and each kit is done in a different tube.

The volume of the sample was recorded. The cells were then pelleted and washed twice with PBS (phosphate buffer saline). The cells were then resuspended to the original volume with PBS. Preparation of the sample took approximately 1 hour.

Six tubes suitable for reading in the flow cytometer were taken, A, B, C, D E and F. Sample (100 μl) was put in each of five of the tubes A-E. In tube F 50 μl of diluted sample (1:20) was placed. Tube A was the control. Tube B was used to measure cell motility. Tetramethylrhodamine (TMR, 0.25 μM) was added to the sample (100 μl) and incubated for 10 minutes at room temperature. The sample was then washed twice with PBS and resuspended with PBS (100 μl). FL-2 was then read using the flow cytometer to determine cell motility.

To determine viability, the sample in Tube C was incubated for 30 minutes at room temperature with Dichlorfluorescein dye (100 μM) and washed twice with PBS. The pellet was resuspended with PBS (100 μl) and the FL-1 was read on the flow cytometer.

Tube D was used to measure the number of white blood cells and immature sperm cells (ISC). Anti CD-45 FITC was added (1 μg/tube) to the sample (100 μl) and incubated for 30 minutes at room temperature. The sample was then washed twice with PBS/Tween 20 (0.05%) and the pellet was resuspended with PBS (100 μl). The white blood cell count and ISC were then measured on the flow cytometer.

Tube E was used to measure the level of anti-sperm antibodies bound to cells. Anti Human IgG,A,M—FITC (5 μg/tube) was added to the sample (100 μl) and incubated for 30 minutes at room temperature. The sample was then washed twice with PBS/Tween (0.05%), the pellet resuspended with PBS (100 μl) and the anti-sperm antibodies measured in the flow cytometer.

Cell count was determined in Tube F by adding 50 μl of FITC beads that contain approximately 20000 beads and the FL-1 was then read over 20 sec on the flow cytometer. Number of cells counted was calculated from the number of beads counted. For example in 20 seconds 23450 cells were counted and 3038 FITC-beads. The number of cells in 50 μl of a 1:20 dilution is 23450:3038×20000×20=154377.88. Therefore, there are 61 million cells per ml. To calculate the number of sperm cells in the sample, the white blood cells and the I.S.C must be subtracted from the number of cells per ml.

The flow cytometer was calibrated with the control sample by reading it through the green fluorescent detector FL-1 and the orange fluorescent detector FL-2 and 0%-3% background for FL-1 (FITC) and FL-2 (TMR) was obtained. Reading by the flow cytometer took 5 minutes.

The results were as follows (raw data not shown):

| Tube No. | Type of test | Result |
|---|---|---|
| A | Control of background | background 0.85 |
| | fluorescence-FL-1 | |
| C | % of viable cells | 99.25% |
| D | WBC | 2.3 million/ml |
| E | % of cells with bound anti- | 3.79% |
| | sperm antibodies | |
| F | cell count | 58.7 million/ml |

The percentage of motile cells in semen was measured with fluorescent dye such as tetramethylrhodamine, in which the dye staining of the cells correlates to the cell's energy. Motile cells are stained and some macrophage cells. After 10 minutes of incubation of cells with tetramethylrhodamine (0.25 μM) the cells were washed twice and the pellet was resuspended with PBS (100 μl) and read by the flow cytometer.

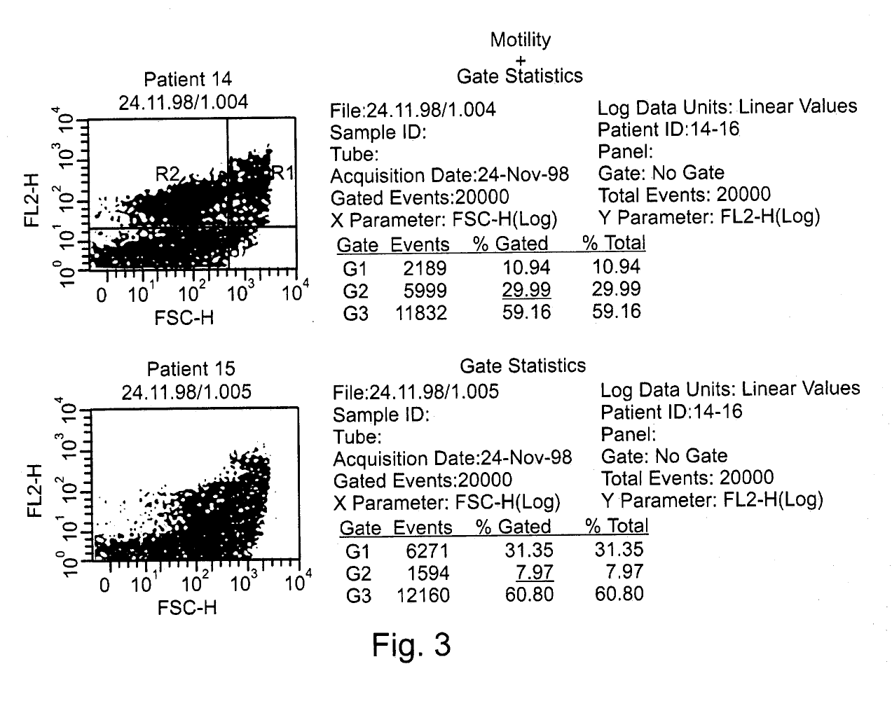

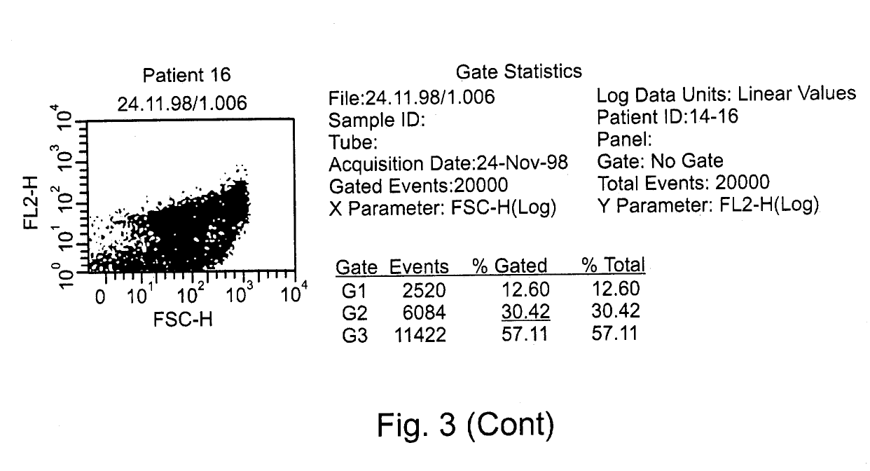

FIG. 3 shows analysis of the motility of 3 semen samples. In gate G1 the cells were bigger in size than sperm cells, in gate G2 were the motile sperm cells and in gate G3 were non-motile cells. The percentage of motile cells in sample A was 29.99%, in sample B was 7.97% and in sample C, 30.42%.

Sperm morphology was also measured. Morphology can be determined based on the pattern of Acridine Orange dye staining. Acridine Orange was added to the sperm cells at a final concentration of 2.5 μM. After 10 minutes incubation at room temperature, followed by two PBS washes, the cells were resuspended to 100 μl in PBS and read by flow cytometer.

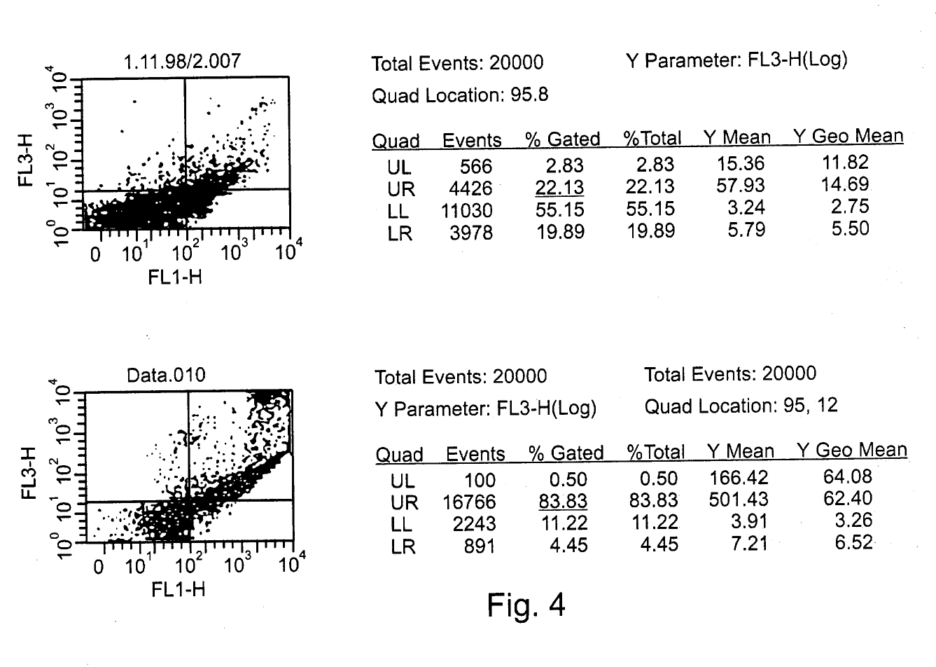

It can be seen from FIG. 4 that sperm cell samples with abnormal morphology or normal morphology have different Acridine Orange staining patterns. In a sperm sample with normal morphology, more than 65% of the cells are in the upper right (UR) window, as was the case in sample 2 (83.83%). In the case of abnormal morphology, less than 65% are in this window, as was found in sample 1 (22.13%).

Identification of sperm antibodies in the cervical mucus

The test for identification of antisperm antibodies in cervical mucus is highly specific, as it only identifies specific antibodies to the sperm antigens. Non specific binding sites are blocked with a blocking solution and therefore there is no identification of antibodies bound to the cells in a non specific way such as fragment Fc′ of the antibody.

To test the presence of sperm antibodies in the neck of the cervix, the sperm cells of the male partner undergo treatment for removal of antibodies (specific and nonspecific), by washing them in a solution of low pH, for this example low pH includes pH 1-7, but preferably pH 3-5. Non-specific binding sites are blocked with a blocking solution and the cells are incubated with liquified cervical mucus from the female. The next step is incubation of sperm cells with fluorescent anti human immunoglobulins (IgG,A,M) for 30 minutes at room temperature, after which the cells are washed and read by flow cytometer. Reading the results of the test in a flow cytometer enables determination of the percentage of cells with antibodies bound to total cell count, which is an important parameter to estimate the effect of the antibodies on decreasing fertility.

The test is very sensitive and identifies antibodies from the three classes IgG, A and M.

### EXAMPLE 2

Detection of antisperm antibodies in cervical mucus

The test for identification of antisperm antibodies in cervical mucus is highly specific. A suitable volume which for a typical sample size is 1 ml of a neutral washing solution such as 0.15M Hepes at pH 7.2 is added to the sperm cell pellet. The cells are resuspended and a suitable volume of treated solution such as 1 ml of 0.2M Hepes at pH 3-5 is added. The cells are incubated for an appropriate amount of time, which in the present example is three minutes. A suitable amount of stop solution, which for a typical sample size is 2 ml of a basic solution such as 0.1 M Hepes at pH 11 is added and the cells are centrifuged.

The pellet is resuspended with an appropriate volume of a suitable blocking solution such as 1 ml of 0.15M Hepes with 5% goat serum and incubated for a suitable amount of time, which in the present example is fifteen minutes at room temperature. The cervical mucus is treated prior to the assay. Treatment involves liquefying the cervical mucus with a suitable reagent such as bromelain 100 μg/ml in a neutral washing solution such as 0.15M at pH 7.2. In the present example one fifth of the sample volume of the liquefied cervical mucus is added to the sperm cells and incubated for thirty minutes at 37° C. The cells are centrifuged and the pellet is resuspended with a suitable amount of fluorescent rabbit anti human Ig in PBS. for a suitable amount of time which in the present example is eight minutes. The cells are centrifuged and the pellet is resuspended with a suitable volume of a neutral washing solution such as 0.25 ml of 0.15M Hepes at pH 7.2. The assay can then be read.

The positive control in the present example is sperm cells with bound antibodies (fixed with formalein) and the negative control is sperm cells without antibodies (fixed with formalein).

Identification of Chlamydia Trachomatis infection in cervical mucus and seminal plasma and determining fertility hormone levels such as LH, FSH and Testosterone in serum

The principle of identification is the binding of specific primary antibodies (monoclonal) to Chlamydia or hormones to beads and their reaction with the test sample. In the next stage (after washing), specific secondary antibodies identify antigens at other sites than those identified by the primary antibodies and bind biotin that is added to the test tube. In the following step, fluorescent straptavidin is added and binds to beads that are labeled by the biotin as positive. The sensitivity of the test is increased by amplifying the positive labeled with fluorescent dye.

### EXAMPLE 3

Identification of Chlamydia Trachomatis infection in seminal plasma and cervical mucus

This experiment is performed to identify infection in seminal plasma and cervical mucus. The cervical mucus or seminal plasma is treated prior to the assay. This is done by adding a suitable reagent to liquefy the cervical mucus or seminal plasma such as bromelain 100 μg/ml in 0.15M Hepes at pH 7.2. A suitable volume, such as one fifth of the sample volume is added and incubated under suitable conditions, such as thirty minutes at 37° C.

Antibodies, specific to Chlamydia trachomatis, are coupled onto beads. These beads are added to the clinical sample and incubated under suitable conditions, for example for thirty minutes at 37° C. The beads are centrifuged and the pellet resuspended with a suitable volume which for a typical sample size is 2 ml of a neutral washing solution such as 0.15M Hepes at pH 7.2. This is repeated twice and the beads are resuspended in a suitable volume which for a typical sample size is 0.1 ml of a neutral washing solution such as 0.15M Hepes at pH 7.2.

Biotinated antibodies that are specific to Chlamydia trachomatis are added and incubated under suitable conditions, which in the present example is thirty minutes at 37° C. The beads are centrifuged and the pellet resuspended with a suitable volume which for a typical sample size is 2 ml of a neutral washing solution such as 0.15M Hepes at pH 7.2. This is repeated twice and the beads are resuspended in a suitable volume, such as 0.1 ml and fluorescent streptavadin is added. This is followed by incubation under appropriate conditions, such as thirty minutes. Fluorescent antibodies directed to streptavidin are added and incubated for 30 minutes at room temperature in the dark. The beads are centrifuged and the pellet is resuspended with a suitable volume, which for a typical sample size is 0.1 ml of a neutral washing solution such as 0.15M Hepes at pH 7.2. The assay can then be read. Positive controls are high level, medium level and low level fluorescent micro sphere beads such as latex beads and negative controls are non-fluorescent micro sphere beads such as non-fluorescent latex beads.

This same protocol can be applied to detection of other genital infections, the only difference being the specification of the antibodies. The same principle is behind the assay to determine gonadotropin levels in sera samples. Identification of FSH is performed as follows:

### EXAMPLE 4

Specific example of identification of FSH

The principle of identification of hormone levels, such as FSH is the binding of specific primary antibodies (monoclonal) with the hormone eg. FSH to beads and their reaction with the test sample.

The system was calibrated with known amounts of FSH. The physiological concentrations of FSH in the serum of women aged 18-55 lie in the following ranges: Women before menopause, with a normal cycle: 10 mIU/ml, women at the ovulation peak: 20-30 mIU/ml and women after menopause: 30-80 mIU/ml. Based on repetitive data obtained with known amounts of FSH (Tubes A, B, C, D and F) within the range 0-100 mIU/ml, a calibration curve was drawn.

Tested serum (Tubes G and H, 100 μl) was incubated for 1 hour at 37° C. with (approximately 5000/tube) beads coupled with antibodies to the tested hormone. Neutral washing solution (0.15M hepes, 2 ml, pH 7.2) was added to the tube and centrifuged to pellet the beads. The washing was repeated and the beads were resuspended with 0.15M Hepes at pH 7.2 containing 1 μg of biotinated monoclonal antibodies specific to FSH. After 30 minutes of incubation, followed by two successive washes, fluorescent streptavidin was added and incubated for 20 minutes. This was followed by addition and incubation with fluorescently labeled goat anti rabbit for 20 minutes. After 3 washes the test sample was read by a flow cytometer and compared to the calibration curve to establish the exact level (mIU/ml) of FSH in the tested sample.

The results are shown in Table 1 (raw data not shown):

| Tube No. | % Of Staining | FSH conc. mIU/ml |
|---|---|---|
| A | 2.3 | 1 |
| B | 6.05 | 5 |
| C | 10.22 | 15 |
| D | 13.36 | 25 |
| F | 25.38 | 50 |
| G | 12.46 | 22 |
| H | 23.92 | 43.5 |

Increasing the rate of success of IVF and IUI

The described protocol is more specific than existing kits, due to the specially designed solutions, which avoid the false positive results evident in the existing kits. Three kits, a stand alone kit to detect sperm-bound antibodies, a device for removal of white blood cells from semen and separation of the motile fraction of sperm cells and removal of sperm-bound antibodies can be used in an unautomated way.

### Example 5

Kit 1—Stand alone kit to detect sperm-bound antibodies

The following stand alone kit can be used to detect sperm-bound antibodies. A suitable amount of sperm cells, which in the present example is about 10 million is washed three times with a suitable volume which for a typical sample size is 2 ml of a neutral washing solution such as 0.15M Hepes at pH 7.2 and 0.001% detergent NP-40. This is done by centrifuging and resuspending the cell pellet. The cell pellet is resuspended with a suitable volume, which for a typical sample size is 0.2 ml of a neutral blocking solution such as 0.15M Hepes at pH 7.2 and 5% rabbit serum and incubated under suitable conditions, such as thirty minutes at 37° C.

A suitable volume of micro sphere beads such as blue latex beads coated with rabbit anti human Ig (F(ab) fragment of rabbit Ig) is added and incubated under the appropriate conditions, such as thirty minutes at 37° C. Sperm-bound micro sphere beads such as latex beads can be seen under a light microscope. The beads are bound if gently flicking off the cover slide does not interfere with the binding. The percentage of sperm-bound micro sphere beads such as latex beads from total number of cells can be calculated. The positive controls with known percentage are read to verify the results.

### EXAMPLE 6

Kit 2—Device for removal of white blood cells from semen and separation of the motile fraction of sperm cells

The device described in the example removes white blood cells from semen and separates the motile fraction of sperm cells. The method is as follows: After seminal liquidation, seminal plasma is separated from cells by centrifuging and resuspension of the cell pellet, with a suitable volume which for a typical sample size is 0.5 ml of neutral solution such as 0.15M Hepes at pH 7.2. The sperm sample is washed and reduced to a suitable volume for example 0.5 ml and placed in the central sample compartment of the device. The fluid touches the sperm sample at two points. The motile sperm will move into the viscous fluid, whereas the immobile sperm cannot. Subsequently the motile sperm can be separated from the sperm sample. The sample is incubated for a suitable time under suitable conditions such as one hour at 37° C. Solution is then collected from both sides of the tube and this is the motile fraction.

### EXAMPLE 7

Kit 3—Removal of sperm-bound antibodies

This experiment removes sperm-bound antibodies. Cell pellet such as 20 million cells is resuspended with a neutral washing solution such as 0.05 ml of 0.15M Hepes at pH 7.2. A ratio of about 40 million sperm cells to 0.1 ml of neutral washing solution is used in the cell treatment. Acidic solution such as 0.05 ml of 0.2M Hepes at pH 2-5 is added to the sperm cells which in a typical sample is about 20 million and incubated under suitable conditions such as for one minute at room temperature. Basic stop solution such as 0.15 ml of 0.2M Hepes at pH 11 and neutral washing solution such as 1 ml of 0.15M Hepes at pH 7.2 is added and the sample centrifuged. The cell pellet is resuspended with a neutral washing solution such as 0.5 ml of 0.15M Hepes at pH 7.2 and a reagent to increase motility of cells such as hyaluronic acid and a reagent to prevent free radical production such as ferulic acid. The sample is incubated under appropriate conditions such as at 37° C. for 1 hour. The level of sperm-bound antibodies can be tested both before and after treatment to check all antibodies have been removed by using the kit for detection of sperm-bound antibodies detailed previously.

Testing sperm cells ability to bind to zp-3

The principle of this test is binding of sperm cells to fluorescent micro sphere beads such as latex beads coated with zp-3 peptides. As a control the ability of sperm cells to bind to these micro sphere beads such as latex beads will be tested in the presence of anti-zp-3 antibodies and with sperm cells that undergo acrosome reaction. Results showing non binding of sperm cells to micro sphere beads such as latex beads are a basis for a negative prediction of success and direct the couple to ICSI treatment as a first choice because lack of binding indicates a low probability for successful IVF and IUI.

Currently available tests require actual, whole oocytes and donor sperm cells for the control, and need highly skilled technical staff. By contrast, the test of the present invention is simple and easy to perform and can be performed both independently and with a flow cytometer.

### EXAMPLE 8

Specific example of determining sperm cells ability to bind to ZP-3 by using a light microscope

The principle of this test is binding of sperm cells to dyed micro sphere beads such as latex beads coated with zp-3 peptides.

Approximately half a million to a million cells of the tested sample are added to tubes A and B. Tube B is a negative control tube. A reagent to induce capacitation reaction of sperm cells, such as BSA 3% is added to Tube A and B for 1 hour at 37° C.

Red dyed beads coated with peptide of the ZP-3 are added to tube A in an appropriate amount. Red dyed beads coated with BSA are added to tube B. To Tube C, the positive control, beads coated with rabbit antibodies to ZP-3 and red dyed beads coated with ZP-3 peptides are added. After 1 hour incubation at 37° C. followed by 2 washes the pellet of each tube is resuspended with 0.5 ml of PBS.

Drops from each tube are placed on slides and viewed under a light microscope. The control slides B and C are compared with slides from tube A. This is done by flicking the cover slide gently to ensure the beads are bound to sperm cells. In tube C the positive control, undyed beads-red beads will be seen. In the case of results where tube A shows no binding and the control tubes give the expected results, the tested male partner is directed to intracytoplasmic sperm injection (ICSI) treatment as a first choice. Lack of binding is indicative of a low probability of successful IVF and IUI.

### EXAMPLE 9

Specific example of determining sperm cells ability to bind to ZP-3 by flow cytometer

Sperm cells (100 μl) were added to tube A (test sample) and to Tube B (control). PBS (100 μl) and BSA (6%) were added to Tubes A and B and incubated for 1 hour at 37° C. Dichlorofluoresein (50 mM) and red fluorescent beads coated with ZP-3 peptides (3 μl) were added to tube A. To Tube B, the control, dichlorofluorescein 50 mM and red fluorescent beads coated with BSA (3 μl) were added and incubated for a further hour at 37° C. After two washes with PBS/Tween 20 (0.05%) the pellets were resuspended with PBS (100 μl) and read by flow cytometer (FL1/FL3 Dots plot).

According to the control tube reading, four gates were defined: Gate 1 (G1) is the red population (only beads), Gate 2 (G2) is the green population (viable sperm cells), Gate 3 (G3) is the unstained population and Gate 4 (G4) is the red-green population (sperm cells that ZP-3 beads are coupled to).

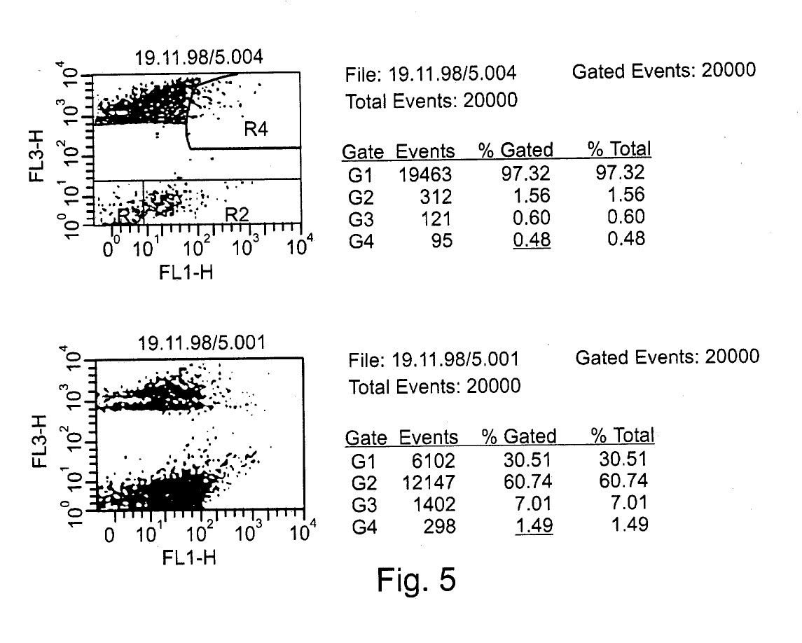

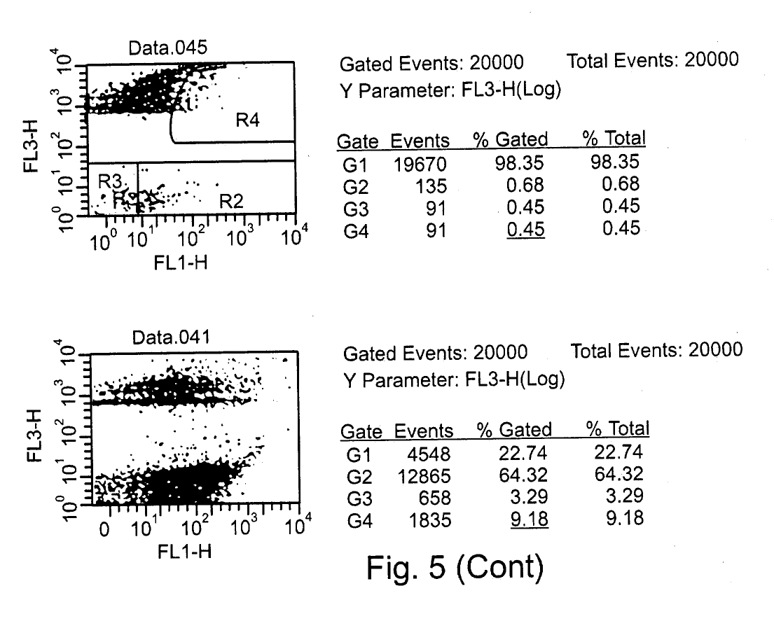

The results from tested samples are shown in FIG. 5. FIG. 5 shows that in sample A the control tube 1 contains beads coated with BSA and incubated with cells. The sample in tube 1 gave a background reading of 0.48%. In tube 2 this percentage was 1.49%, indicating a low binding ability. In the sample in tube 3 the control tube gave a background reading of 0.45%. The percentage of binding in tube 4 was 9.18% indicative of normal binding ability.

### EXAMPLE 10

Testing the ability of sperm cells to undergo acrosome reaction

Two additional tubes A and B were then used to obtain an acrosome reading. To Tube A containing sperm cells (100 μl) was added a reagent which induces acrosome reaction in vitro, progesterone (10 μg/ml) ,Ca²⁺ (10 μM) and platinic chloride (100 μM). This was incubated for 15 minutes at 37° C. and then washed with PBS. The pellet was resuspended with PBS (100 μl).

In Tube B sperm cells (100 μl) were placed and to both Tubes A and B was added 1 μg of monoclonal antibody anti-CD46-PE (orange fluorescent). CD46 was exposed only after completion of the acrosome reaction.

After two washes with PBS/Tween 20 (0.05%) the pellet was resuspended with PBS (100 μl) and the results read on the flow cytometer in FL-2.

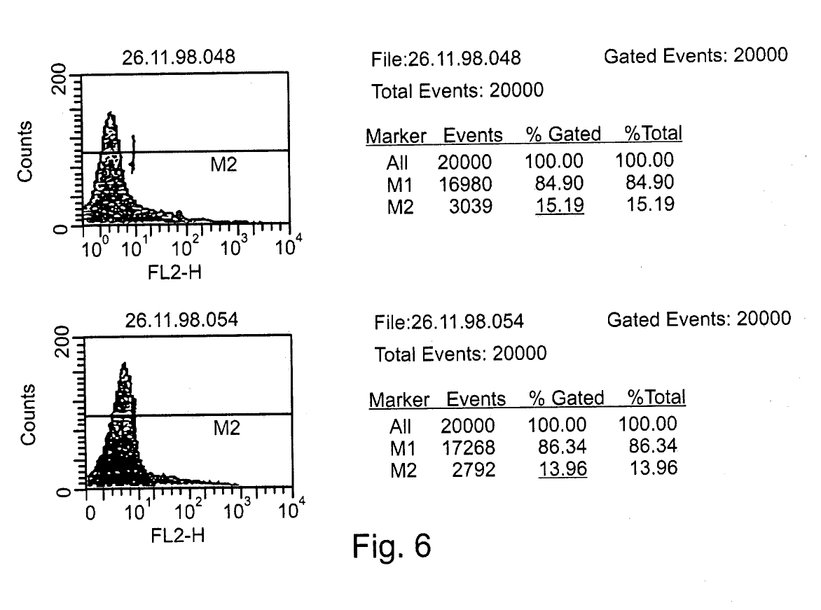

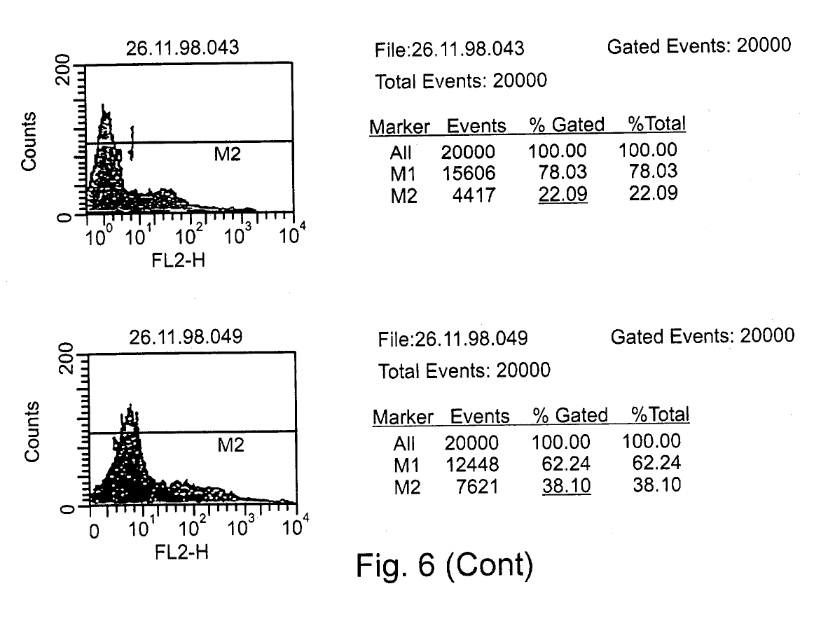

The results can be seen in FIG. 6. FIG. 6 shows that in Tube A1 the percentage of cells that underwent acrosome reaction was 22.09%. In tube B1 sperm cells of the same sample were incubated with a specific reagent to induce in vitro acrosome reaction. The percentage of cells that underwent acrosome reaction was higher 38.10%. In tube A2 (different subject), the percentage of cells that underwent acrosome reaction was 15.19%. B2 shows the results of in vitro induction of acrosome reaction, which in this case was unsuccessful, 13.96%.

### EXAMPLE 11

Example of identification of ZP-3 autoantibodies in female sera

This experiment was performed to identify ZP-3 autoantibodies in female sera. The method was as follows: The tested serum and negative serum (without ZP-3 antibodies) diluted 1:100 with PBS were incubated with beads coupled with ZP-3 protein and with beads without ZP-3 for 1 hour at 37° C. After two washes the pellet was resuspended with PBS (100 μl/tube) and anti-human IgG,A,M (1 μl) labeled with fluorescent dye was added and incubated for 30 minutes at room temperature in the dark. After a further 2 washes, the pellet was resuspended with PBS (100 μl) and the results in the flow cytometer.

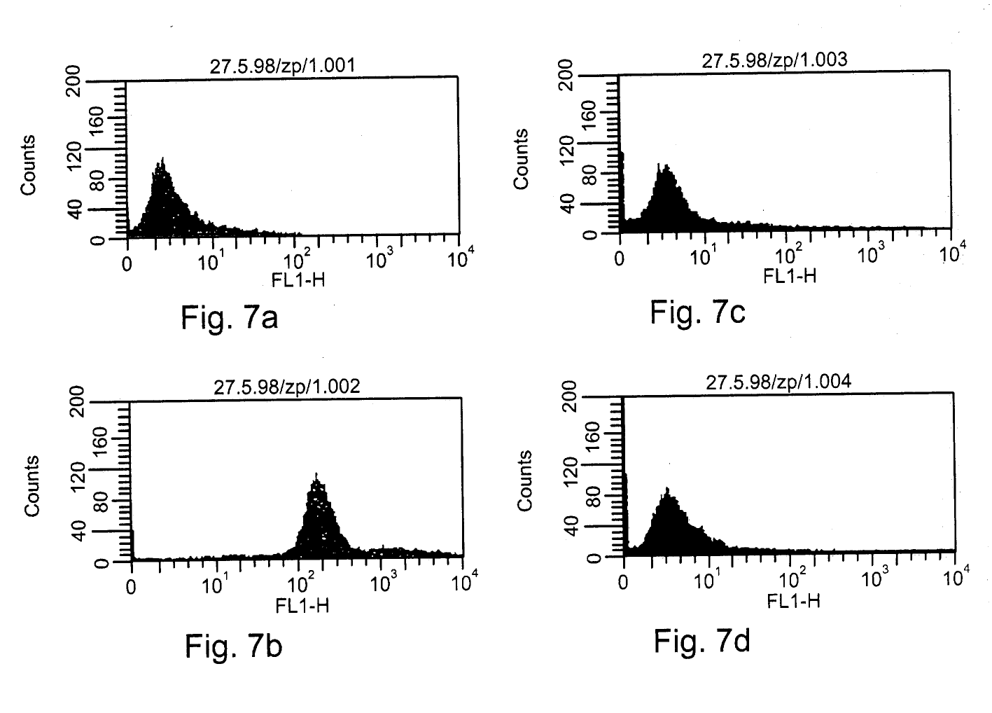

In FIG. 7:

7A=Negative serum incubated with beads coated with ZP-3.

7B=Tested serum incubated with beads coated with ZP-3.

7C=Negative serum incubated with the same kind of beads but uncoupled with ZP-3.

7D=Tested serum incubated with the same kind of beads but uncoupled with ZP-3.

The results show that in the tested serum, anti ZP-3 autoantibodies are present and bound to ZP-3. The bound antibodies are labeled with FITC-anti human antibodies (B) in contrast to the negative control serum in which no human antibodies were coupled, ZP-beads (A).

7C and 7D show that the binding in B is specific to ZP-3, as without ZP-3 there is no binding of antibodies from the tested serum.

### EXAMPLE 12

Specific Example of eluted antisperm antibodies from the sperm surface

There has been demonstrated a clear association between sperm surface antibodies and reduced likelihood of pregnancy. The following example is a method to separate antibody free sperm. This example of the embodiment of the principles of the invention is not limiting.

ELISA wells were coated with rabbit-anti human IgG IgA (10 μg/ml) in carbonate buffer (pH9.8, 0.1M) 100 μl/well and incubated for 1 hour at 37° C. The plate was washed three times with phosphate buffer saline (PBS) (pH 7.2, 0.1M) containing 0.05% Tween -20. Blocking solution (5% rabbit serum in PBS) was added 150 μl/well and the plate was incubated for 1 hour at 37° C. The plate was washed as previously, three times with PBS (pH 7.2, 0.1M) containing 0.05% Tween -20. Sperm cells before and after treatment to elute sperm antibodies were added to each well and each sample was added to six wells. Positive control was sperm cells that were incubated (before the ELISA) with human serum that was found to contain anti sperm antibodies, and negative control was sperm cells with no antibodies bound and PBS. 0.5 million cells/well in PBS (150 μl) was added and the plate incubated for 1 hour at 37° C. The plate was washed as before. Peroxidase labeled anti human IgG was added to three wells of each sample and peroxidase labeled anti human IgA to the other three wells with the same sample. The plate was incubated for 1 hour at 37° C. After three washes peroxidase substrate was added (o-phenylenediamine-OPD) and the optical density was measured by an ELISA reader.

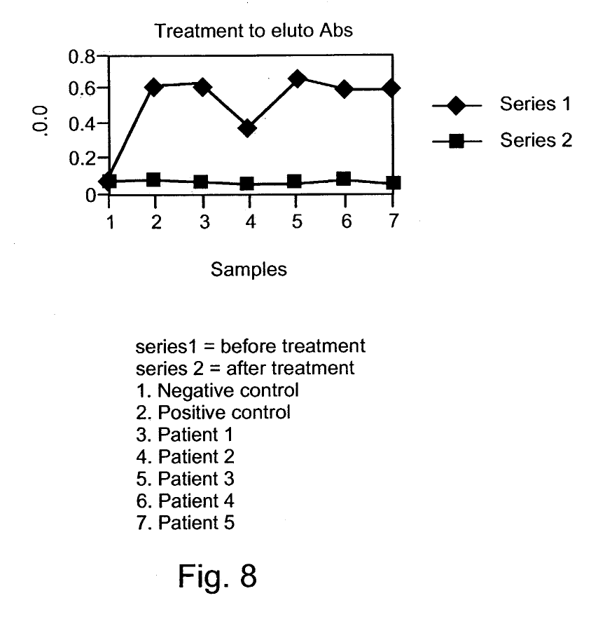

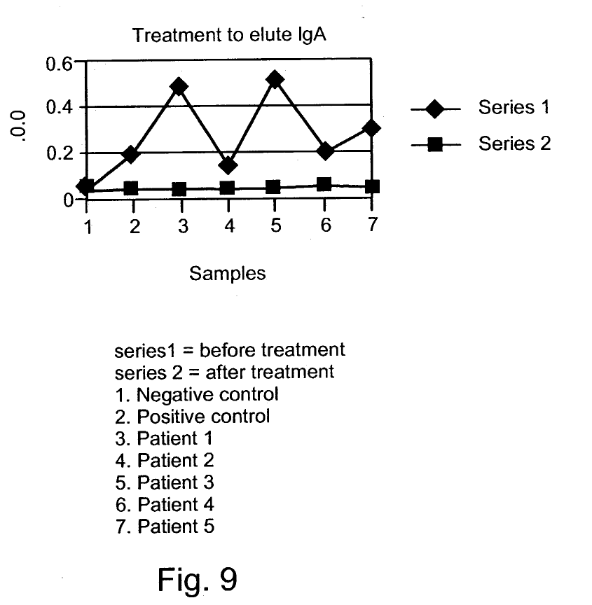

The effect of the treatment to elute IgG and IgA from sperm cells in five patients are shown in FIG. 8 and FIG. 9. By testing the level of sperm-bound antibodies before and after treatment, it was found that all antibodies had been removed. The viability and motility of sperm cells before and after the treatment to remove sperm-bound antibodies were compared. The treatment shows no effect or only a slight effect (1-2%) on both viability and motility.

It will be appreciated that the above examples and descriptions are intended only to serve as examples, and that many other embodiments are possible within the spirit and the scope of the present invention.

What is claimed:

1. A semi-automated fertility system for assessing the fertility of a couple, the couple consisting of a male partner and a female partner, comprising;
(a) a cervical smear including cervical mucus and at least one serum sample from the female partner;
(b) at least one semen sample and at least one serum sample from the male partner;
(c) a fertility kit for determining at least one fertility affecting factor, said fertility kit being used to perform a fertility test, said fertility kit including at least one reagent, such that said reagent is able to react with a sample selected from the group consisting of a cervical smear and serum sample from the female partner and, a semen sample and a serum sample from the male partner, to form a reaction product; and
(d) a flow cytometer, such that said flow cytometer is able to analyse said reaction product to determine said fertility factor.

2. The system of claim 1, wherein a plurality of tests can be performed on a single sample of the group consisting of, at least one female cervical smear, female cervical mucus, at least one female serum sample, at least one male semen sample and at least one male serum sample.

3. The system of claim 1, wherein said sample from the male partner is said semen sample, and said reagent is a viscous solution, such that motility of sperm in said sample is determined according to movement of said sperm through said viscous solution.

4. The system of claim 3, wherein said viscous solution includes a dye.

5. The system of claim 3 further comprising a device for measuring sperm motility in a sample of sperm, said device comprising:
(a) a sample compartment;
(b) at least one channel; and
(c) a barrier separating said sample compartment from said at least one channel, such that said sperm must cross over said barrier from said sample compartment to reach said at least one channel.

6. The system of claim 1, wherein said sample from the male partner is said semen sample, and said reagent is a dye to identify live cells, such that said fertility test determines a number of live cells.

7. The system of claim 6, wherein said dye includes dichlorfluorescein.

8. The system of claim 1, wherein said sample from the male partner is said semen sample, and said reagent is a morphology gate system comprising at least one gate such that said fertility test determines sperm cell morphology according to an ability of said sperm cells to enter through said at least one gate.

9. The system of claim 8, wherein said access is determined by geometry of said gate.

10. The system of claim 8, further comprising a dye.

11. The system of claim 10, wherein said dye is acridine orange.

12. The system of claim 1, wherein said sample from the male partner is said semen sample, and said reagent comprising:
(a) a solution including anti human antibodies conjugated with fluorescent dye, said anti human antibodies binding to an antibody present in cells of said semen sample; and
(b) a second solution including a dyed label, said dyed label binding to said anti human antibodies, such that antibodies bound to sperm are detected;
such that said fertility test is detection of sperm-bound antibodies.

13. The system of claim 12, wherein said reagent comprises:
(a) a solution to remove non-specific antibodies; and
(b) a second solution to block non-specific antibody binding sites on the sperm surface.

14. The system of claim 1, wherein said sample from the male partner is said semen sample, and said reagent is fluorescent micro sphere beads coated with zp-3 antibodies, and said fertility test is ability of said sperm to bind to said beads.

15. The system of claim 1, wherein said sample comprises said cervical smear of the female partner and said semen sample of the male partner, and said reagent comprises at least one antibody specific to at least one infectious agent of the genitalia, such that said fertility test is detection of said infectious agent in cervical smear and semen sample.

16. The system of claim 15 further comprising:
(a) polystyrene micro sphere beads coated with an antibody specific to an infectious agent;
(b) at least one biotin labeled antibody specific to said infectious agent, said biotin conjugate binding to said beads;
(c) a streptavidin protein, said protein binding to biotin; and
(d) a fluorescent labeled dye, said dye binding to said antibody.

17. The system of claim 15, wherein said sample comprises said cervical smear of the female partner and said semen sample of the male partner, and said reagent comprises at least one antibody specific to Clamydia trachomatis, such that said fertility test is detection of Chlamydia trachomatis in cervical smear and semen sample.

18. The system of claim 17 further comprising:
(a) polystyrene micro sphere beads coated with an antibody specific to Clamydia trachomatis;
(b) at least one biotin labeled antibody specific to Chlamydia trachomatis, said biotin labeled antibody binding to said beads;
(c) a streptavidin protein, said protein binding to biotin; and
(d) a fluorescent labeled dye, said dye binding to said antibody.

19. The system of claim 1, wherein said sample comprises said serum sample of the female partner and said serum sample of the male partner such that said fertility test is detection of hormone levels in serum sample.

20. The system of claim 19 further comprising:
(a) at least one polystyrene micro sphere bead coated with antibodies specific for the hormone to be tested;
(b) at least one biotin labeled antibody, said antibody binding to said hormone;
(c) a streptavidin protein, said protein binding to biotin; and
(d) a fluorescent labeled dye, said dye binding to said antibody.

21. The system of claim 1, wherein said fertility test is the ability of sperm cells to undergo acrosome reaction.

22. The system of claim 1, wherein said fertility test is sperm cell count and white blood cell count.

23. A semi-automated system for assessing diagnostic factors, comprising;
(a) at least one cell and body fluid sample;
(b) a kit for determining at least one diagnostic factor, said kit being used to perform a diagnostic test, said kit including at least one reagent, such that said reagent is able to react with at least one cell and body fluid sample to form a reaction product; and
(c) a flow cytometer, such that said flow cytometer is able to analyse said reaction product to determine said diagnostic factor.

24. The system of claim 23, wherein said diagnostic factor is hormone level.

25. The system of claim 23, wherein said diagnostic factor is the identification of antigens of any component of an infectious agent.

26. The system of claim 23, wherein said diagnostic factor is a fertility factor.

27. A method for detecting sperm-binding antibodies in cervical mucus of the female partner comprising the steps of:
(a) washing semen sample of the male partner in a solution of low pH to remove specific and non specific antibodies;
(b) incubating the semen sample of the male partner in a solution to block non specific binding sites in the semen sample;
(c) incubating treated semen sample of the male partner with cervical mucus of the female partner;
(d) incubating mixture of said treated semen sample of the male partner and cervical mucus of the female partner with anti human antibodies bound to fluorescent dye; and
(e) detecting results in flow cytometer.

28. A method for predicting success of IVF and IUI treatment, comprising the steps of:
(a) washing and capacitation of sperm sample;
(b) incubating said sperm sample with fluorescently labeled beads coated with peptides of the oocyte- membrane;
(c) washing said sperm cells; and
(d) detecting sperm cells bound to oocyte membrane peptide to predict success of IVF and IUI treatment.

29. The method of claim 28, wherein said prediction of success of IVF and IUI treatment is determined by visual observation of a dye.

30. A method of collecting motile sperm cells from a sample of sperm, comprising the steps of:
(a) providing a device for measuring sperm motility in a sample of sperm, said device including;
(i) a sample compartment;
(ii) at least one channel; and
(iii) a barrier separating said sample compartment from said at least one channel, such that said sperm must cross over said barrier from said sample compartment to reach said channel;
(b) filling said channels of said device with a viscous solution;
(c) putting said sample in said sample compartment of said device; and
(d) collecting motile sperm cells from said channels of said device.

31. The method of claim 30, further comprising separating white blood cells by magnetic separation with magnetic beads coated with anti CD-45 antibodies.

32. A method of removal of sperm bound antibodies from semen comprising the steps of:
(a) forming a cell pellet by centrifugation of the semen;
(b) adding an acidic solution to said cell pellet to remove antisperm antibodies; and
(c) resuspending cell pellet in a mixture of washing solution, reagent to increase cell motility and a reagent to prevent free radical production to obtain semen without sperm bound antibodies.

33. The method of claim 32, wherein said reagent to increase cell motility includes hyaluronic acid.

34. The method of claim 32, wherein said reagent to prevent free radical production includes ferulic acid.

35. A method for increasing success of IVF treatment and IUI treatment, comprising the steps of:
(a) removing white blood cells and separating motile sperm cells from semen by:
(i) providing a device, for separation of motile sperm cells from non-motile material, said non-motile material including white blood cells, in a sample of sperm, said device comprising;
(I) a sample compartment;
(II) at least one channel; and
(III) a barrier separating said sample compartment from said at least one channel, such that said sperm must cross over said barrier from said sample compartment to reach said channel;
(ii) filling said channels of said device with a viscous solution;
(iii) mixing semen with magnetic beads coupled with anti CD45;
(iv) putting said sample in said sample compartment and incubating; and
(v) collecting motile sperm cells from said channels;
(b) removing sperm bound antibodies by:
(i) forming a cell pellet by centrifugation;
(ii) adding an acidic solution to remove antisperm antibodies; and
(iii) resuspending cell pellet in a mixture of washing solution, reagent to increase cell motility and a reagent to prevent free radical production.

36. A device for measuring sperm motility in a sample of sperm, comprising;
(a) a sample compartment;
(b) at least one channel; and
(c) a barrier separating said sample compartment from said at least one channel, such that said sperm must cross over said barrier from said sample compartment to reach said at least one channel.

37. The device of claim 36, wherein said at least one channel contains a viscous fluid.

38. The device of claim 37, wherein said viscous fluid contains at least one dye, such that the sperm are able to contact said dye upon reaching said at least one channel.
